(19) United States
(12) Patent Application Publication
(10) Pub. No.: US 2016/0155228 A1
(43) Pub. Date: Jun. 2, 2016

(54) MEDICAL IMAGE GENERATION APPARATUS, METHOD, AND PROGRAM

(71) Applicants: KABUSHIKI KAISHA TOSHIBA, Minato-ku (JP); National Institute of Radiological Sciences, Chiba-shi (JP)

(72) Inventors: Yukinobu SAKATA, Kawasaki (JP); Ryusuke HIRAI, Shinagawa (JP); Kyoka SUGIURA, Kawasaki (JP); Yasunori TAGUCHI, Kawasaki (JP); Tomoyuki TAKEGUCHI, Kawasaki (JP); Shinichiro MORI, Sakura (JP); Fumi MARUYAMA, Miura (JP)

(73) Assignees: KABUSHIKI KAISHA TOSHIBA, Minato-ku (JP); National Institute of Radiological Sciences, Chiba-shi (JP)

(21) Appl. No.: 14/953,224

(22) Filed: Nov. 27, 2015

(30) Foreign Application Priority Data
Nov. 28, 2014 (JP) 2014-241667

(51) Int. Cl. G06T 7/00; G06K 9/46; A61B 6/00; G06T 11/00

(52) U.S. Cl. CPC G06T 7/0012; G06T 7/0081; G06T 11/00; G06K 9/46; A61B 19/50; A61B 6/5205; G06T 2207/30096; G06T 2207/10116; G06T 2200/04; A61B 2019/507

(57) ABSTRACT

A medical image generation apparatus includes: a three-dimensional image acquisition unit that acquires a three-dimensional image in which a space including a patient is captured; an imparting unit that imparts, to each of voxels constituting the three-dimensional image, a living-body likelihood coefficient indicating a likelihood of being a living-body region of the patient; an updating unit that updates a luminance value of the voxel in which the imparted living-body likelihood coefficient shows a given value, through predetermined processing; a virtual viewpoint setting unit that sets a virtual viewpoint for transforming the three-dimensional image into a two-dimensional radiation image; and a radiation image generation unit that calculates a luminance value of a pixel constituting the radiation image based on the luminance value of the voxel existing along a line connecting each of the corresponding pixels and the virtual viewpoint.

### CROSS-REFERENCE TO RELATED APPLICATIONS

This application is based upon and claims the benefit of priority from Japanese Patient application No. 2014-241667, filed on Nov. 28, 2014, the entire contents of each of which are incorporated herein by reference.

### BACKGROUND OF THE INVENTION

1. Field of the Invention

Embodiments of the present invention relates to a medical image generation technology for virtually generating a radiation image from a three-dimensional image.

2. Description of the Related Art

A treatment technology of radiating a particle beam to malignant tumors such as cancer is attracting attention because the technology has excellent features such as high treatment effects, few adverse effects, and reduction of a burden on a human body.

A particle beam made incident on the body of a patient loses kinetic energy during passing, and when it is lowered to a certain velocity, it stops suddenly and a high dose called a Bragg peak is generated.

Due to a high dose generated at a pinpoint as described above, it is possible to shoot only cancer cells to kill them, whereby effects on normal cells can be minimized.

Accordingly, in a treatment apparatus using a particle beam, it is required to accurately set the sight of the beam radiated to the affected area, in order not to damage normal tissue.

As such, before starting radiation of the beam, the position of an affected area is specified by X-ray observation or the like, the position and the angle of the movable bed on which the patient is placed are adjusted appropriately, and the affected area is positioned accurately within the radiation range of the beam.

Such positioning is performed by matching a radiation image (DRR: Digitally Reconstructed Radiograph) virtually generated from a three-dimensional image used in treatment planning performed in advance, and the X-ray observation image (for example Patent Document WO2008/021245).

However, in the conventional method, if the three-dimensional image includes a bed, a restraint, or the like having an attenuation value which is the same as that of human tissue, it is impossible to separate the bed or the restraint from a VOI (Volume of Interest) of the patient in the three-dimensional image. As such, a DRR of the patient in which such a bed or a restraint is captured is generated.

Further, in particle beam radiation therapy, a time lag of several weeks may be caused from the time when a three-dimensional image of a patient for the above treatment planning is captured until the time when the affected area is irradiated with the particle beam.

Furthermore, there is also a case where the positional relations between the patient and the bed and the restraint thereof differ between the time of capturing the three-dimensional image for treatment planning and the time of radiating the beam.

As such, there is a problem that deterioration in matching accuracy between the radiation image (DRR) generated from the three-dimensional image (VOI), and the X-ray observation image captured at the time of radiating the beam, may not be avoidable.

### SUMMARY OF THE INVENTION

Embodiments of the present invention have been made in consideration of such a situation. An object of the present invention is to provide a medical image generation technology capable of generating a radiation image in which only a patient is extracted, even in the case where an object (a bed, a restraint, or the like) having an attenuation value which is the same as that of human tissue is included in a three-dimensional image.

A medical image generation apparatus according to an embodiment of the present invention comprises a three-dimensional image acquisition unit that acquires a three-dimensional image in which a space including a patient is captured; an imparting unit that imparts, to each of the voxels constituting the three-dimensional image, a living-body likelihood coefficient indicating the likelihood of being a living-body region of the patient; a voxel luminance value updating unit that updates a luminance value of the voxel in which the imparted living-body likelihood coefficient shows a given value, through predetermined processing; a virtual viewpoint setting unit that sets a virtual viewpoint for transforming the three-dimensional image into a two-dimensional radiation image; and a radiation image generation unit that calculates a luminance value of a pixel constituting the radiation image based on the luminance value of the voxel existing along a line connecting each of the corresponding pixels and the virtual viewpoint.

According to the embodiments of the present invention, an object of the present invention is to provide a medical image generation technology capable of generating a radiation image in which only a patient is extracted, even in the case where an object (a bed, a restraint, or the like) having an attenuation value which is the same as that of human tissue is included in a three-dimensional image.

### BRIEF DESCRIPTION OF THE DRAWINGS

FIG. 1 is a block diagram showing a medical image generation apparatus according to a first embodiment of the present invention;

FIG. 2 is a block diagram showing a medical image generation apparatus according to a second embodiment;

FIGS. 3A, 3B, and 3C show radiation images generated by setting a weighting factor W so as to emphasize a given voxel luminance value V in a three-dimensional image capturing a head;

FIG. 4 is a block diagram showing a medical image generation apparatus according to a third embodiment;

FIG. 5 is a table showing model information;

FIGS. 6A, 6B, 6C, and 6D are illustrations showing a procedure of deriving a living-body likelihood coefficient using bed/restraint model information and body contour information of a patient;

FIG. 7 is a block diagram showing a medical image generation apparatus according to a fourth embodiment; and

FIG. 8 is a flowchart explaining a medical image generation method and a medical image generation program according to embodiments.

### DETAILED DESCRIPTION OF THE PREFERRED EMBODIMENTS

### First Embodiment

Hereinafter, an embodiment of the present invention will be described based on the accompanying drawings.

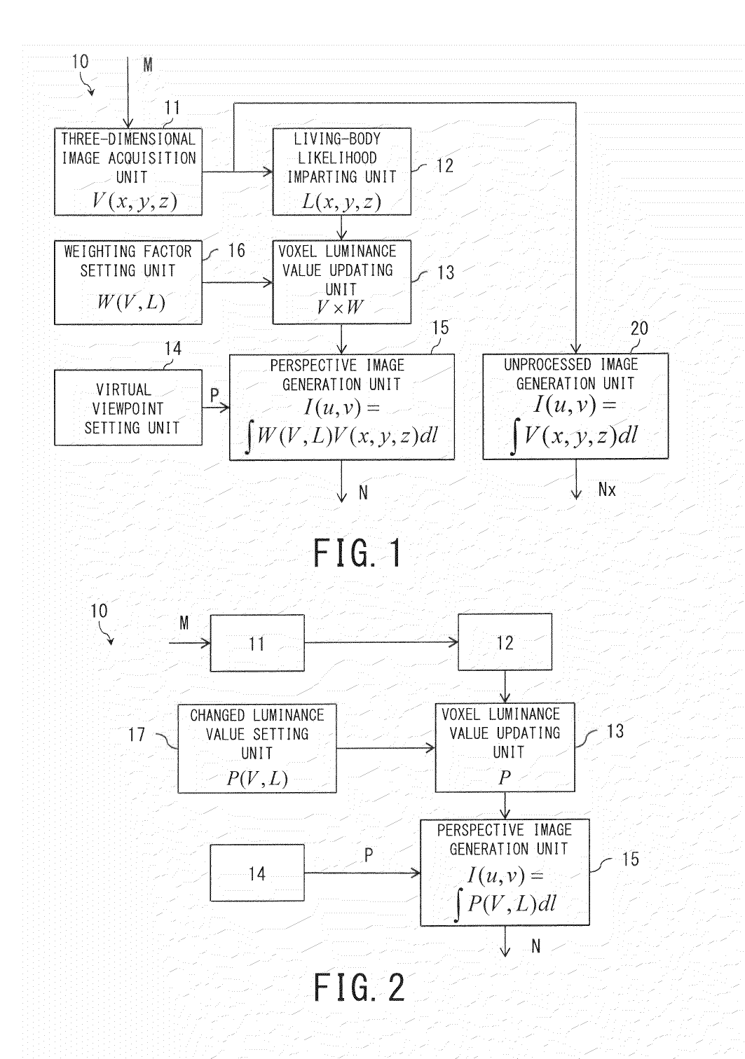

As shown in FIG. 1, a medical image generation apparatus 10 according to a first embodiment includes a three-dimensional image acquisition unit 11 which acquires a three-dimensional image M capturing a space including a patient, an imparting unit 12 which imparts, to each of the voxels constituting the three-dimensional image M, a living-body likelihood coefficient L(x,y,z) showing the likelihood of being a living region of the patient, an updating unit 13 which updates a luminance value V(x,y,z) of a voxel in which the imparted living-body likelihood coefficient L shows a given value through predetermined processing, a virtual viewpoint setting unit 14 which sets a virtual viewpoint P for transforming a three-dimensional image M into a two-dimensional radiation image N, and a radiation image generation unit 15 which calculates a luminance value I(u,v) of a pixel constituting the radiation image N based on the luminance value V(x,y,z) of the voxel existing along a line connecting each of the corresponding pixels and the virtual viewpoint P. The radiation image includes, for example, a fluoroscopic image, a radiograph, or a perspective image.

The medical image generation apparatus 10 is further includes a weighting factor setting unit 16 which sets a weighting factor W(V,L) corresponding to a luminance value V of a voxel and a living-body likelihood coefficient L.

The updating unit 13 multiplies the luminance value V of the voxel in which the imparted living-body likelihood coefficient L shows the given value, by a corresponding weighting factor W(V,L), to thereby update the luminance value V of the voxel.

A three-dimensional image M acquired by the three-dimensional image acquisition unit 11 is a three-dimensional image of the inside of a patient body captured by an X-ray CT scanner, for example. Besides, an image captured by an MRI apparatus may be adopted as a three-dimensional image M. There is no limitation on such an image provided that the image shows a three-dimensional structure of a patient body.

It should be noted that the three-dimensional image M includes not only the inside of a patient body but also a bed on which the patient is placed, a restraint for restraining the patient and the bed, and the like.

The three-dimensional image acquisition unit 11 may receive such a three-dimensional image M from a medical image capturing device of various types, or from an image server, a medium such as a CD, DVD, or the like, a network storage, or the like.

The voxels constituting the three-dimensional image M are uncertain whether they constitute an object such as a bed, a restraint, or the like, constitute a living-body region of the patient, or constitute a space around the patient.

The living-body likelihood coefficient imparting unit 12 calculates, for each of the voxels, a living-body likelihood coefficient L showing the likelihood of being a living-body region of the patient, and associates it with positional information (x,y,z) thereof.

Calculation of such a living-body likelihood coefficient is performed by extracting a group region of voxels having continuity in luminance values V(x,y,z), and based on at least one type of information among average luminance value, size, shape, positional relation, and the like of each group region, imparting a living-body likelihood coefficient L of the same value to the voxels constituting each group region.

Another calculation method may be used. In such a method, a dictionary in which a living-body likelihood of a voxel is calculated from images having living body/non-living body labels prepared in advance, according to a feature quantity extracted from a surrounding pattern of each pixel, has been studied. Then, a living-body likelihood is calculated by applying the dictionary to each voxel of the three-dimensional image M. A method of calculating a living-body likelihood coefficient is not limited, particularly.

A living-body likelihood coefficient L(x,y,z) may be represented by a binary value (living body/non-living body) for the cases of a living-body region and a non-living body region other than it.

A living-body likelihood coefficient L(x,y,z) may also be represented by a ternary value (living body/intermediate/non-living body), or by discretized values.

A living-body likelihood coefficient L(x,y,z) may also be represented by continuous values or discretized values as a section in which an upper limit value indicates the case of definitely being a living-body region and a lower limit value indicates the case of definitely being a non-living body region other than it.

Further, it is also possible to divide the section of a living-body likelihood coefficient L(x,y,z) represented by continuous values or discretized value by an arbitrary value, and associate one of the divided section with a living-body region and associate the other with a non-living body region.

The updating unit 13 updates a luminance value V(x,y,z) of a voxel in which the imparted living-body likelihood coefficient L shows a given value, through predetermined processing.

In the predetermined processing, an easy one is that a voxel, to which a value “1” indicating that the living-body likelihood coefficient L is a “living body” is imparted, is updated with the luminance value V being the same value, while a voxel, to which a value “0” indicating that the living-body likelihood coefficient L is a “non-living body” is imparted, is updated with the luminance value V being a zero value.

Through this processing, objects such as a bed, a restraint, and the like are eliminated from the three-dimensional image M, and it is corrected to a three-dimensional image configured of a living-body region of the patient and the surrounding space.

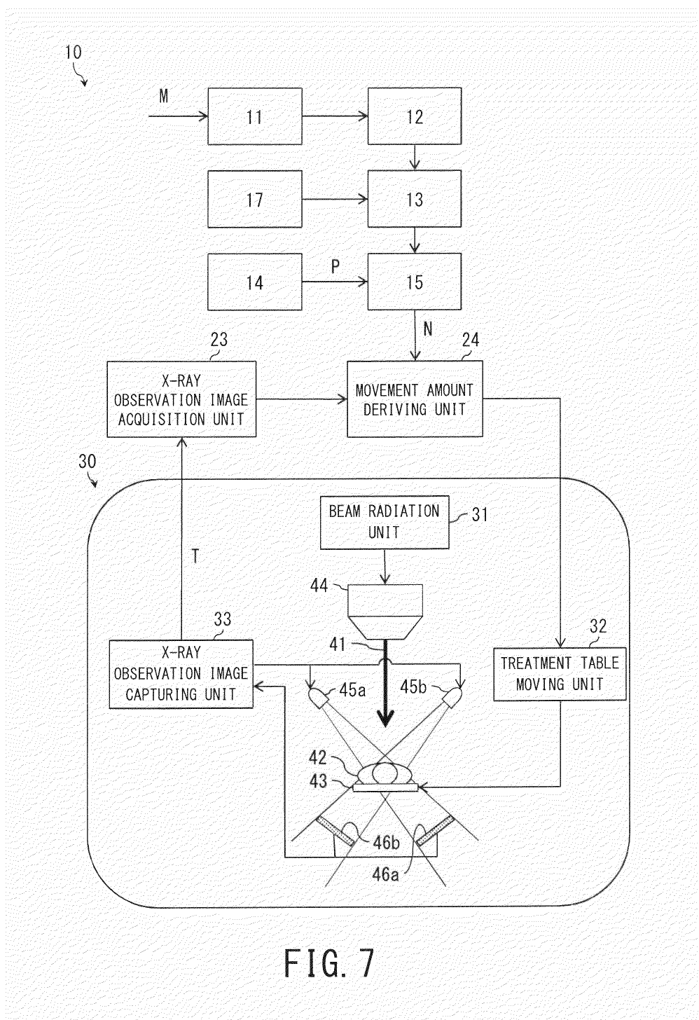

The virtual viewpoint setting unit 14 sets a virtual viewpoint P for transforming a three-dimensional image M into a two-dimensional radiation image N. Specifically, the virtual viewpoint is a muzzle 44 of a beam 41 of a particle beam radiation therapy apparatus 30 (FIG. 7) described below. The position of the virtual viewpoint P is determined in consideration of the incident position and the direction of the beam 41 with respect to the patient 42 placed on the treatment table 43.

The radiation image generation unit 15 calculates a luminance value I(u,v) of a pixel constituting the radiation image N based on the luminance value V(x,y,z) of the voxel existing along a line connecting each of the corresponding pixels and the virtual viewpoint P.

It should be noted that a method of calculating a luminance value I(u,v) of a pixel is not limited, particularly. Besides integration of the luminance values V(x,y,z) of the voxels along the line, there is a case of calculating it as a product of exp such as I(u,v)=Πexp(V(x,y,z)).

Thereby, objects such as a bed and a restraint are eliminated, and a radiation image N in which only a living-body region of the patient is seen through is generated.

It should be noted that an unprocessed image generation unit 20 is a unit which generates a radiation image Nx of a plane three-dimensional image M in which luminance value of a voxel is not updated.

The weighting factor setting unit 16 is configured such that when a living-body likelihood coefficient L is represented by a binary value (living body/non-living body), a weighting factor W(V,1) corresponding to a luminance value V of a voxel showing a living body and a weighting factor W(V,0) corresponding to a luminance value V of a voxel showing a non-living body are set separately.

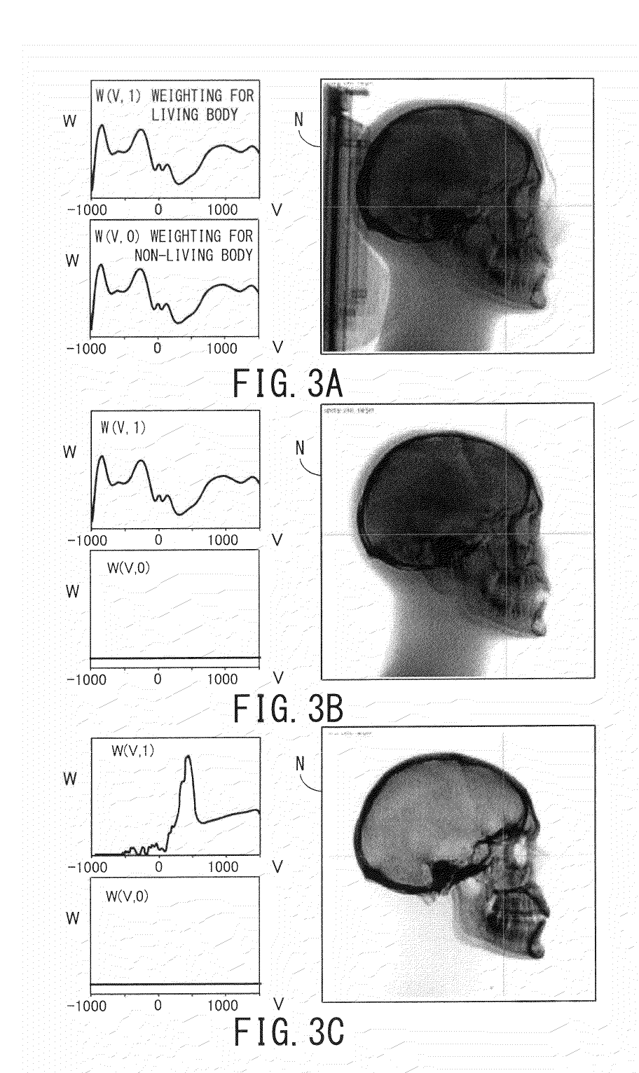

FIGS. 3A, 3B, and 3C show radiation images N of a head seen through from a side face direction by setting weighting factors W so as to emphasize the luminance values V of voxels constituting a skull.

FIG. 3A shows a radiation image in the case where distributions of weighting factors W with respect to luminance values V of voxels are the same in the weighting factors W(V,1) for a living body and the weighting factors W(V,0) for a non-living body.

In this case, while the skull is emphasized in the radiation image N, a living-body region other than the skull and images of a bed and a restraint are also included.

FIG. 3B shows a radiation image in the case where regarding the weighting factors W(V,0) for a non-living body, weighting factors W with respect to luminance values V of voxels are in a flat distribution at zero.

In this case, while the skull is emphasized in the radiation image N, a living body region other than the skull is included.

FIG. 3C shows a radiation image in the case where regarding the weighting factors W(V,1) for a living body, distribution of the weighting factors W is set so as to further emphasize the voxel luminance values V of the skull.

In this case, a radiation image N in which the living-body region other than the skull is eliminated and only the skull is emphasized is obtained.

The weighting factor setting unit 16 sets distribution graphs of the weighting factors W of the number corresponding to the number of given values (two in FIG. 3, that is, living body and non-living body) of the living-body likelihood coefficient L.

On one hand, when the living-body likelihood coefficient L is represented by continuous values, the upper limit value and the lower limit value of the living-body likelihood coefficient L are standardized to be 0 and 1, and a living-body likelihood coefficient L of any value between them is assumed to be taken, for example.

In this case, the weighting factor setting unit 16 sets a weighting factor W(V,L) based on the following Expression (1).

W(V,L)=L×W(V,1)+(1−L)×W(V,0) (1)

The radiation image generation unit 15 calculates a luminance value I(u,v) of a pixel constituting the radiation image N based on multiplies a product of a luminance value V(x,y,z) of the voxel existing along a line connecting each of the corresponding pixels and the virtual viewpoint P, by the corresponding weighting factor W(V,L).

According to the first embodiment, even in the case where a three-dimensional image includes an object (bed, restraint, or the like) having an attenuation value which is the same as that of human tissue, it is possible to generate a radiation image in which only a patient is extracted.

### Second Embodiment

Next, a second embodiment of the present invention will be described with reference to FIG. 2. In FIG. 2, parts having configurations or functions common to those in FIG. 1 are denoted by the same reference signs, and the description thereof is not repeated herein.

A medical image generation apparatus 10 according to the second embodiment is further includes a changed luminance value setting unit 17 which sets a changed luminance value P(V,L) corresponding to a luminance value V of a voxel and a living-body likelihood coefficient L.

Then, the updating unit 13 updates the luminance value V of a voxel in which the imparted living-body likelihood coefficient L shows a given value, to a corresponding changed luminance value P.

Further, the radiation image generation unit 15 calculates a luminance value I(u,v) of a pixel constituting the radiation image N based on the changed luminance value P(V,L) of a voxel existing along a line connecting each of the corresponding pixels and the virtual viewpoint P.

The changed luminance value setting unit 17 sets changed luminance values P of the number corresponding to the product of the number of living-body likelihood coefficients L and the number of luminance values L of the voxels.

If the living-body likelihood coefficient L is represented by continuous values, the changed luminance value setting unit 17 sets a changed luminance value P(V,L) based on the following Expression (2).

P(V,L)=L×P(V,1)+(1−L)×P(V,0) (2)

On the other hand, considering the case where a changed luminance value P(V,L) takes a binary value, that is, P(V,1) in the case of being a living body and P(V,0) in the case of being a non-living body, by setting a constant as in the following Expression (3) and allowing the constant to be sufficiently small, it is possible to obtain a radiation image in which the living-body region of the patient is emphasized.

P(V,1)=V,P(V,0)=const. (3)

According to the second embodiment, even in the case where a three-dimensional image includes an object (bed, restraint, or the like) having an attenuation value which is the same as that of human tissue, it is possible to generate a radiation image in which only a patient is extracted.

### Third Embodiment

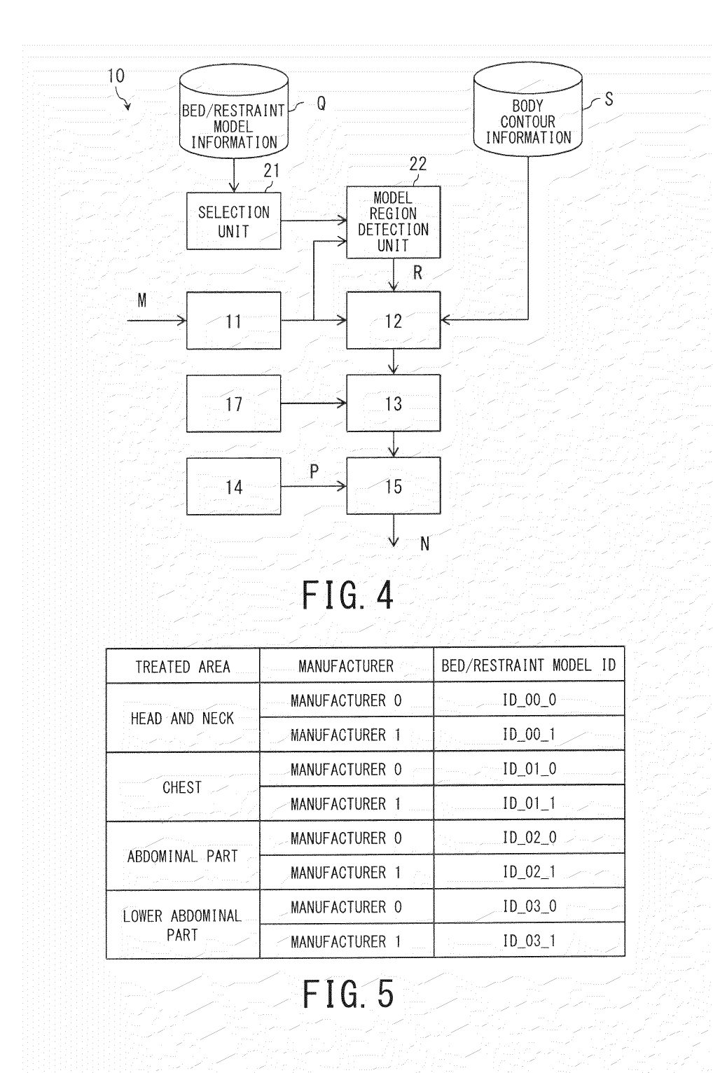

Next, a third embodiment of the present invention will be described with reference to FIG. 4. In FIG. 4, parts having configurations or functions common to those in FIG. 1 or 2 are denoted by the same reference signs, and the description thereof is not repeated herein.

A medical image generation apparatus 10 according to the third embodiment is further includes an accumulation unit which accumulates a model information Q of a shape of a bed on which a patient is placed when a three-dimensional image M is captured or a restraint for restraining the patient and the bed, a selection unit 21 which selects an arbitrary model from the plurality of accumulated model information Q, and a detection unit 22 which detects a model region R of voxels matching the selected model, from the acquired three-dimensional image M.

Then, the living-body likelihood coefficient imparting unit 12 imparts a living-body likelihood coefficient L (“0”) indicating a non-living body region, to the voxels constituting the detected model region R.

As the bed/restraint model information Q, data in which only a bed or a restraint is captured by a medical image capturing device in a state where a patient is not placed, and data in which the shape of a bed or a restraint is obtained such as CAD data of a bed or a restraint, may be adopted appropriately.

Further, regarding fixtures, as the shape and size thereof differ depending on treated sites or manufacturers, a plurality of models are prepared in advance as shown in FIG. 5 so as to be able to select model information Q to be used according to information of a treated site or a manufacturer.

The medical image generation apparatus 10 according to the third embodiment is further includes an accumulation unit which accumulates body contour information S of a patient set at the time of treatment planning.

Then, the living-body likelihood coefficient imparting unit 12 acquires the body contour information S, and imparts a living-body likelihood coefficient L (“1”) indicating a living-body region, to the inner region of the body contour.

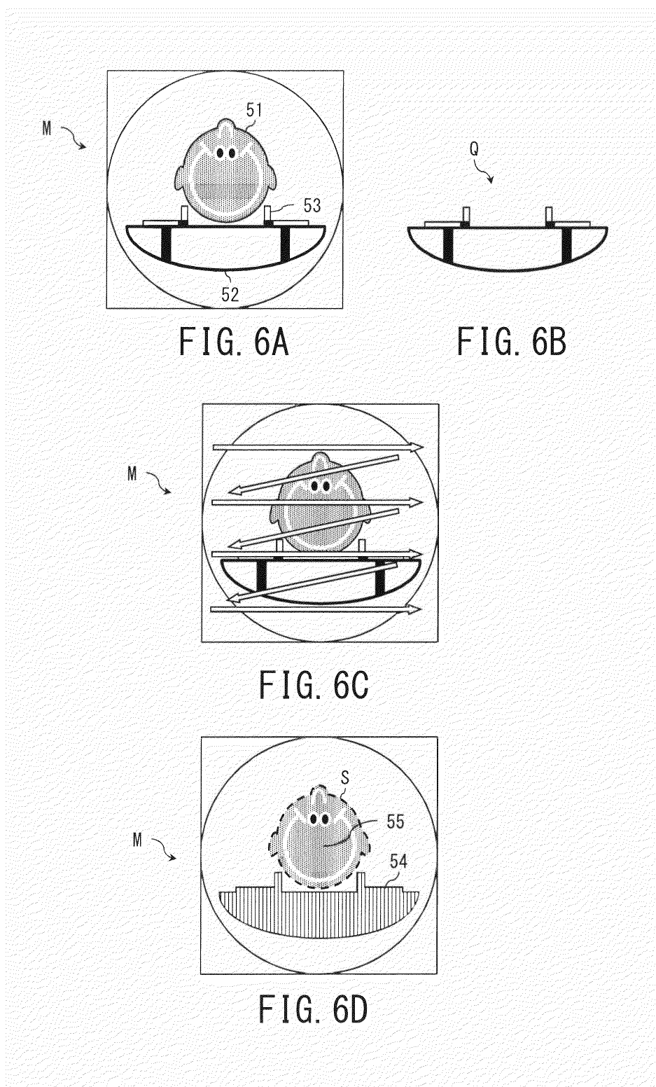

A procedure of deriving a living-body likelihood coefficient using bed/restraint model information Q will be described with reference to FIGS. 6A, 6B, 6C, and 6D.

FIG. 6A shows a three-dimensional image M including a patient 51, a bed 52, and a restraint 53.

FIG. 6B shows bed/restraint model information Q.

As shown in FIG. 6C, by performing raster scanning on the three-dimensional image M, a region corresponding to the model information Q is detected in the three-dimensional image M. The detection may be performed using SSD, SAD, normalized cross correlation, mutual information amount, and the like so as to detect a region of the three-dimensional image M in which a deviation from the model information Q becomes minimum. Further, by performing searching while changing rotation or a scale, it is possible to detect a regions of a bed or a restraint having different orientations or sizes in the three-dimensional image M.

If the bed/restraint model information Q is CAD data, by imaging the CAD data corresponding to the capturing format of the three-dimensional image M, it is possible to perform detection by the same method.

As shown in FIG. 6D, a living-body likelihood coefficient L (“0”) indicating a non-living body region is imparted to a region 54 of the three-dimensional image M corresponding to the model information Q. Then, a living-body likelihood coefficient L (“1”) indicating a living-body region is imparted to an inner region 55 of the body contour information S.

It should be noted that the three-dimensional image M may be handled as a stereoscopic image or a plurality of two-dimensional sliced images.

According to the third embodiment, even in the case where a three-dimensional image includes an object (bed, restraint, or the like) having an attenuation value which is the same as that of human tissue, it is possible to generate a radiation image in which only a patient is extracted with yet higher accuracy.

### Fourth Embodiment

Next, a fourth embodiment of the present invention will be described with reference to FIG. 7. In FIG. 7, parts having configurations or functions common to those in FIG. 1 or 2 are denoted by the same reference signs, and the description thereof is not repeated herein.

Here, the particle beam radiation therapy apparatus 30 is configured to shoot an affected area in the body of the patient 42 with the beam 41 for treatment so as to treat the affected area.

If the beam 41 is a heavy particle beam, when the beam 41 is made incident on the body, kinetic energy is lost during passing, and when it is lowered to a certain velocity, it stops suddenly and a high dose called a Bragg peak is generated. Due to a high dose generated at a pinpoint as described above, it is possible to shoot only cancer cells to kill them, and to minimize effects on normal tissue.

As such, a treatment technology using the beam 41 of a heavy particle beam has excellent features such as high treatment effects, few adverse effects, and reduction of a burden on a human body, with respect to malignant tumors such as cancer.

Regardless of the type of medical beam 41, in the particle beam radiation therapy apparatus 30, it is required to accurately set the sight of the beam 41 radiated to the affected area, in order not to damage normal tissue.

As such, before starting radiation of the beam, the position of an affected area is specified by X-ray observation or the like, the position and the angle of the movable treatment table 43 on which the patient is placed are adjusted appropriately by a moving unit 32, and the affected area is positioned accurately within the radiation range of the beam 41.

The particle beam radiation therapy apparatus 30 is configured of a beam radiation unit 31 which radiates the beam 41 from the muzzle 44, the moving unit 32 which moves the treatment table 43 on which the patient 42 is placed such that the beam 41 aims the affected area, and an image capturing unit 33 which captures an X-ray observation image T of the patient by controlling X-ray generation units 45 (45a, 45b) and X-ray detection units 46 (46a, 46b).

The medical image generation apparatus 10 according to the fourth embodiment is further equipped with an acquisition unit 23 which acquires the X-ray observation image T of the patient captured by the image capturing unit 33, and a deriving unit 24 which derives the amount of movement of the treatment table 43, based on the radiation image N and the X-ray observation image T.

The movement amount deriving unit 24 derives, as an amount of movement, the amount of positional deviation between the radiation image N at the time of planning the treatment and the X-ray observation image T capturing a state where the patient 42 is placed on the treatment table 43 of the particle beam radiation therapy apparatus 30.

The amount of positional deviation is defined by six parameters, namely three-dimensional translation (tx,ty,tz) and rotation (rx,ry,rz).

The six parameters are expressed as the following Expression (4) where a radiation image generated from a three-dimensional image to which arbitrary displacements R and T of rotation and position are given is P(3D_IMG^{RT}), an X-ray observation image is X, and an error between the two images is D(,).

R^{g},T^{g}=arg minD(X,P(3D_IMG^{RT})) (4)

Here, D(,) may take any index if it represents an error between the two images. For example, SSD, SAD, normalized cross correlation, mutual information amount, or the like may be used. When positioning the patient, comparison between the images and update of R and T are performed in turn to thereby derive the final amount of positional deviation.

In the operation performed the movement amount deriving unit 24, as a bed, a restraint, or the like is not captured in the radiation image N, the amount of positional deviation from the X-ray observation image T is derived with high accuracy.

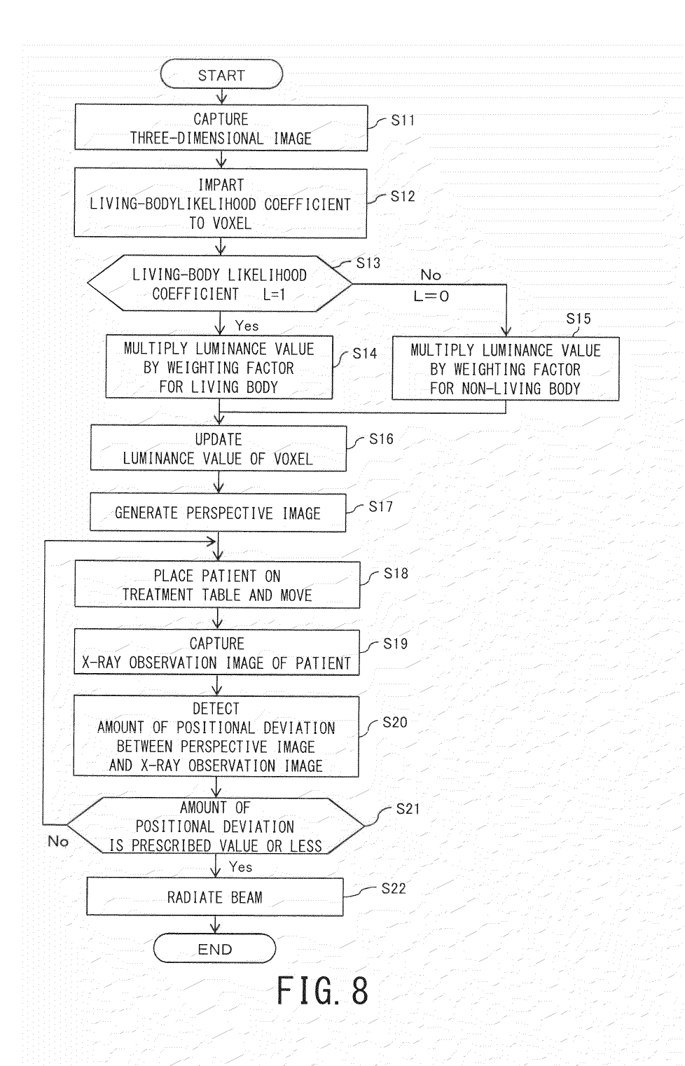

Embodiments of a medical image generation method and a medical image generation program according to the fourth embodiment will be described based on the flowchart of FIG. 8.

A three-dimensional image M of a space including a patient captured by an X-ray CT scanner or the like is acquired (S11). To each of the voxels constituting the three-dimensional image M, a living-body likelihood coefficient L indicating the likelihood of being a living-body region of the patient is imparted (S12).

The luminance value V of a voxel in which the living-body likelihood coefficient L is “1” is multiplied by a weighting factor W(V,1) for a living body (S13 Yes, S14), and the luminance value V of a voxel in which the living-body likelihood coefficient L is “0” is multiplied by a weighting factor W(V,0) for a non-living body (S13 No, S15), whereby the luminance values are updated (S16).

The luminance values of the voxels existing along a line extending from a set virtual viewpoint P are integrated to be the luminance values of pixels and then to generate the radiation image N (S17).

The patient 42 is placed on the treatment table 43 of the particle beam radiation therapy apparatus 30, and the treatment table 43 is moved directly under the muzzle 44 (S18). In this state, the image capturing unit 33 is operated to capture an X-ray observation image P of the patient 42 (S19), and the amount of positional deviation between the radiation image N and the X-ray observation image P is detected (S20).

Then, if the amount of positional deviation exceeds a prescribed value, the amount of movement of the treatment table 43 required for setting the sight of the beam 41 on the affected area is derived, and the treatment table 43 is moved again (S21 No, S18). Then, when the amount of positional deviation becomes the prescribed value or less, the beam 41 is radiated (S21 Yes, S22).

According to the medical image generation apparatus of at least one of the embodiments described above, by imparting a living-body likelihood coefficient to each of the voxels constituting the three-dimensional image of a patient, even in the case where a three-dimensional image includes an object (bed, restraint, or the like) having an attenuation value which is the same as that of a human tissue, it is possible to generate a radiation image in which only a patient is extracted. Further, even in the case where the positional relation between the patient and a bed or a restraint differs between the time of planning and the time of treatment, it is possible to perform positioning with high accuracy by solely focusing on the patient.

While some embodiments of the present invention have been described, those embodiments are shown as examples and are not intended to limit the scope of the invention. These embodiments may be carried out in various other forms, and various omissions, replacements, changes, and combinations can be made within the scope not deviating from the gist of the invention. These embodiments and the variations thereof are included in the scope and the gist of the invention, and are also included in the invention described in the claims and in the scope of the equivalents thereof.

It should be noted that the medical image generation apparatus can be realized by using a general-purpose computer device as basic hardware, for example. This means that the respective function units can be realized by causing a processor installed in a computer device to execute a program. At this time, the medical image generation apparatus may be realized by previously installing the program in the computer device, or may be realized by storing the program in a storage medium such as a CD-ROM or by distributing the program via a network and installing the program in the computer device as required.

What is claimed is:

1. A medical image generation apparatus comprising:
a three-dimensional image acquisition unit that acquires a three-dimensional image in which a space including a patient is captured;
an imparting unit that imparts, to each of voxels constituting the three-dimensional image, a living-body likelihood coefficient indicating a likelihood of being a living-body region of the patient;
an updating unit that updates a luminance value of the voxel in which the imparted living-body likelihood coefficient shows a given value, through predetermined processing;
a virtual viewpoint setting unit that sets a virtual viewpoint for transforming the three-dimensional image into a two-dimensional radiation image; and
a radiation image generation unit that calculates a luminance value of a pixel constituting the radiation image based on the luminance value of the voxel existing along a line connecting each of the corresponding pixels and the virtual viewpoint.

2. The medical image generation apparatus according to claim 1, wherein
the living-body likelihood coefficient is represented by continuous values or discretized values as a section in which an upper limit value indicates a case of definitely being a living-body region and a lower limit value indicates a case of definitely being a non-living body region other than the living-body region.

3. The medical image generation apparatus according to claim 1, wherein
the living-body likelihood coefficient is represented by a binary value indicating a case of being the living-body region and a case of being a non-living body region other than the living-body region.

4. The medical image generation apparatus according to claim 1, further comprising
a weighting factor setting unit that sets a weighting factor corresponding to the luminance value of the voxel and the living-body likelihood coefficient, wherein
the updating unit updates the luminance value of the voxel by multiplying the luminance value of the voxel, in which the imparted living-body likelihood coefficient shows the given value, by a corresponding weighting factor.

5. The medical image generation apparatus according to claim 1, further comprising:
a changed luminance value setting unit that sets a changed luminance value corresponding to the luminance value of the voxel and the living-body likelihood coefficient, wherein
the updating unit updates the luminance value of the voxel in which the imparted living-body likelihood coefficient shows the given value, to the corresponding changed luminance value.

6. The medical image generation apparatus according to claim 1, wherein
the imparting unit extracts a group region of the voxels having continuity in the luminance values, and based on at least one type of information among types of information such as an average luminance value, size, shape, and positional relation of each of the group regions, imparts the living-body likelihood coefficients having a same value to the voxels constituting each of the group regions.

7. The medical image generation apparatus according to claim 1, further comprising:
an accumulation unit that accumulates a model information of a shape of a bed on which the patient is placed when the three-dimensional image is captured or of a restraint for restraining the patient and the bed;
a selection unit that selects an arbitrary model from the plurality of accumulated model information; and
a detection unit that detects, from the acquired three-dimensional image, a model region of the voxel matching the selected model, wherein
the imparting unit imparts the living-body likelihood coefficient indicating a non-living body region, to the voxel constituting the detected model region.

8. The medical image generation apparatus according to claim 1, wherein
the imparting unit acquires body contour information of the patient, and imparts the living-body likelihood coefficient indicating the living-body region to an inner region of the body contour.

9. The medical image generation apparatus according to claim 1, further comprising:
an X-ray observation image acquisition unit that acquires an X-ray observation image of the patient captured by an X-ray image capturing unit provided in a vicinity of a muzzle of a radiation beam; and
a deriving unit that derives an amount of movement of a treatment table in order to position an affected area directly under the muzzle by moving the patient based on the radiation image and the X-ray observation image.

10. A medical image generation method comprising the steps of:
acquiring a three-dimensional image in which a space including a patient is captured;
imparting, to each of voxels constituting the three-dimensional image, a living-body likelihood coefficient indicating a likelihood of being a living-body region of the patient;
updating a luminance value of the voxel in which the imparted living-body likelihood coefficient shows a given value, through predetermined processing;
setting a virtual viewpoint for transforming the three-dimensional image into a two-dimensional radiation image; and
calculating a luminance value of a pixel constituting the radiation image based on the luminance value of the voxel existing along a line connecting each of the corresponding pixels and the virtual viewpoint.

11. A medical image generation program for causing a computer to perform the steps of:
acquiring a three-dimensional image in which a space including a patient is captured;
imparting, to each of voxels constituting the three-dimensional image, a living-body likelihood coefficient indicating a likelihood of being a living-body region of the patient;
updating a luminance value of the voxel in which the imparted living-body likelihood coefficient shows a given value, through predetermined processing;
setting a virtual viewpoint for transforming the three-dimensional image into a two-dimensional radiation image; and
calculating a luminance value of a pixel constituting the radiation image based on the luminance value of the voxel existing along a line connecting each of the corresponding pixels and the virtual viewpoint.
